(19) United States
(12) Patent Application Publication
(10) Pub. No.: US 2025/0221778 A1
(43) Pub. Date: Jul. 10, 2025

(54) POWER SUPPLY CONTROL METHOD, SYSTEM, DEVICE AND MEDIUM FOR SURGICAL ROBOT

(71) Applicant: Precision Robotics (Hong Kong) Limited, Shatin, N.T. (HK)

(72) Inventors: Ping Lai Benny Lo, Shatin, N.T. (HK); Ka King Wong, Shatin, N.T. (HK)

(21) Appl. No.: 18/690,733

(22) PCT Filed: Aug. 17, 2023
(86) PCT No.: PCT/CN2023/113636

§ 371 Date: Dec. 16, 2024

Related U.S. Application Data
(60) Provisional application No. 63/398,550, filed on Aug. 17, 2022.

(51) Int. Cl. A61B 34/30

(52) U.S. Cl. CPC A61B 34/30

(57) ABSTRACT

A power supply control method, a system, device and a medium for a surgical robot. The surgical robot includes a power control module (2) for outputting power, and the control method includes the following steps: reading starting time sequence information of corresponding electrical devices of the surgical robot (S1); and controlling the power control module (2) to supply power to the electrical devices in turn, according to the starting time sequence information (S2). Based on the analysis of different electrical components of the surgical robot, a corresponding electrical control logic is reasonably designed, so that components of the surgical robot system can obtain independent DC power supply. By controlling the power-on time of the electrical components, current surge can be effectively prevented when the electrical components are powered on, so that the surgical robot can effectively work safely and stably.

### CROSS-REFERENCE TO RELATED APPLICATIONS

This application is a National Stage Application under 35 U.S.C. § 371 of PCT Application No. PCT/CN2023/113636, filed Aug. 17, 2023, which claims priority to U.S. Provisional Patent Application No. 63/398,550, filed Aug. 17, 2022, the entire contents of which are incorporated by reference herein.

### TECHNICAL FIELD

The present disclosure relates to the technical field of a surgical robot, in particular to a power supply control method, system, device and medium for a surgical robot.

### BACKGROUND

With the popularity of robotic surgery system, more and more surgical operations are realized by surgical robots. The iterative updating of the function of the surgical robot also makes the device tend to be complicated and refined. There are usually multiple motors in the system. If the power supply unit (PSU) and other devices are powered on at the same time during the working process, a fuse may be blown due to current surge, which will not only affect the normal use of the surgical robot, but even lead to medical accidents, which greatly limits the application of the surgical robot.

### SUMMARY

A technical problem to be solved by the present disclosure is to provide a power supply control method for a surgical robot in order to overcome the defects in the related art. The surgical robot includes a power control module for outputting power, and the control method includes:
reading starting time sequence information of corresponding electrical devices of the surgical robot; and
controlling the power control module to supply power to the electrical devices in turn, according to the starting time sequence information.

Preferably, the power control module includes at least one direct current (DC) power control board and at least one alternating current (AC) power control board. The electrical devices include a DC electrical device and an AC electrical device. The DC power control board is configured to output DC power to the DC electrical device, and the AC power control board is configured to output AC power to the AC electrical device. The step of controlling the power control module to supply power to the electrical devices in turn includes:
controlling the AC power control board and the DC power control board respectively, to supply power to the DC electrical device and the AC electrical device in turn;
where the starting time sequence information is set according to a working parameter of the DC electrical device and the AC electrical device, and the working parameter includes at least one of starting current and rated current.

Preferably, the DC electrical device includes electric equipment corresponding to the surgical robot. The AC electrical device includes a computer device corresponding to the surgical robot.

An order of supplying power to the DC electrical device and the AC electrical device in turn is as follows: firstly, supplying power to the computer device, and then supplying power to the corresponding electric equipment and motorized device.

Preferably, after the surgical robot is started, the step of reading the starting time sequence information of the corresponding electrical devices of the surgical robot is performed.

The present disclosure further provides a power supply control system for a surgical robot, which includes a power control module for outputting power, and the control system includes:
an information reading module, configured to read starting time sequence information of corresponding electrical devices of the surgical robot; and
a power supply control module, configured to control the power control module to supply power to the electrical devices in turn according to the starting time sequence information.

Preferably, the power control module includes at least one DC power control board and at least one AC power control board. The electrical devices include a DC electrical device and an AC electrical device. The DC power control board is configured to output DC power to the DC electrical device, and the AC power control board is configured to output AC power to the AC electrical device.

The power supply control module is configured to control the AC power control board and the DC power control board respectively, to supply power to the DC electrical device and the AC electrical device in turn. The starting time sequence information is set according to a working parameter of the DC electrical device and the AC electrical device, and the working parameter includes at least one of starting current and rated current.

Preferably, the DC electrical device includes electric equipment corresponding to the surgical robot. The AC electrical device includes a computer device corresponding to the surgical robot.

An order of the power supply control module supplying power to the DC electrical device and the AC electrical device in turn is as follows: firstly, supplying power to the computer device, and then supplying power to the corresponding electric equipment and motorized device.

Preferably, the information reading module is called after the surgical robot is started.

The present disclosure further provides an electronic device, which includes a memory, a processor and a computer program stored in the memory and executable on the processor, and when the computer program is executed by the processor, the power supply control method for the surgical robot is realized.

The present disclosure further provides a computer-readable storage medium, on which a computer program is stored, and when the computer program is executed by a processor, the power supply control method for the surgical robot is realized.

The positive progress effect of the present disclosure is in that: by providing a power supply control method, system, device and medium for a surgical robot, based on the analysis of different electrical components of the surgical robot, a corresponding electrical control logic is reasonably designed, so that the components of the surgical robot system can obtain independent DC power supply. By controlling the power-on time of the electrical components, current surge can be effectively prevented when the surgical robot is powered on, so that the surgical robot can effectively work safely and stably, and the present disclosure has the significance of industrial popularization.

### BRIEF DESCRIPTION OF THE DRAWINGS

FIG. 1 is a flowchart of a power supply control method for a surgical robot in Embodiment 1 of the present disclosure.

FIG. 2 is a schematic diagram of starting time sequence information for the power supply control in Embodiment 1 of the present disclosure.

FIG. 3 is a schematic diagram of modules of a power supply control system for a surgical robot in Embodiment 2 of the present disclosure.

FIG. 4 is a block diagram of a structure of an electronic product according to Embodiment 3 of the present disclosure.

### DETAILED DESCRIPTION

In order to explain the technical solution of the embodiments of this specification more clearly, the accompanying drawings needed in the description of the embodiments will be briefly introduced below. Obviously, the accompanying drawings in the following description are only some examples or embodiments of this specification. For those skilled in the art, this specification can be applied to other similar situations according to these accompanying drawings without creative work. Unless it is obvious from the linguistic context or otherwise stated, the same reference signs in the accompanying drawings represent the same structure or operation.

As shown in this specification, the words “a”, “an”, “one” and/or “the” do not refer to the singular, but may also include the plural. Generally, the terms “include” and “contain” only imply the inclusion of clearly identified steps and elements, and these steps and elements do not constitute an exclusive list, and a method or device may also contain other steps or elements.

### Embodiment 1

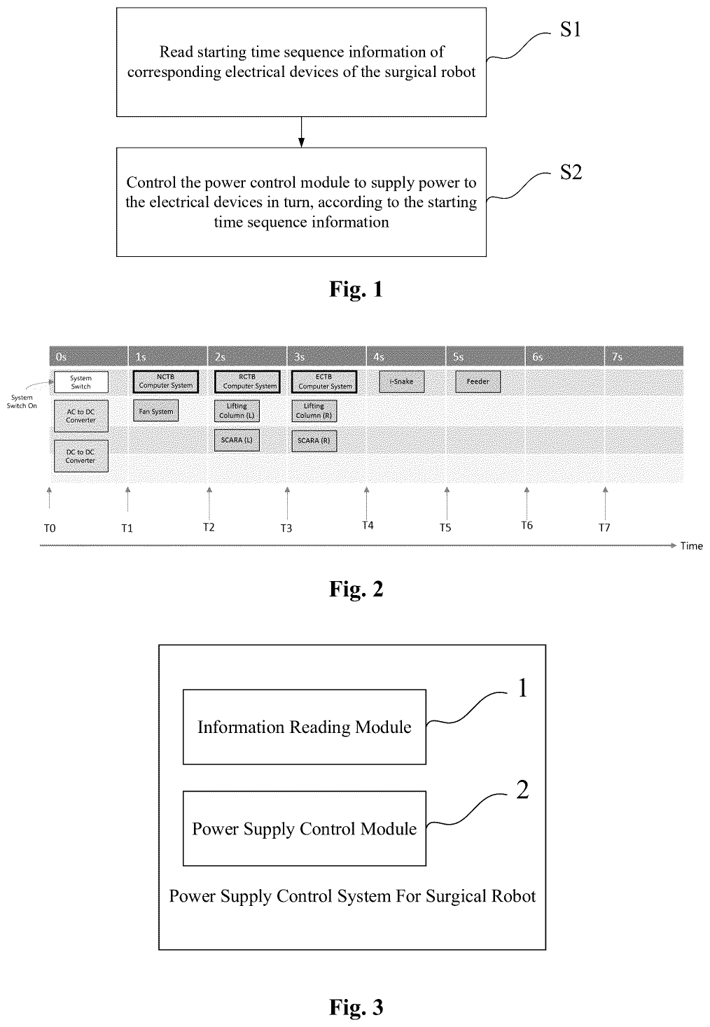

As shown in FIG. 1, this embodiment specifically provides a power supply control method for a surgical robot. The surgical robot includes a power control module for outputting power, and the control method includes:
S1, reading starting time sequence information of corresponding electrical devices of the surgical robot; and
S2, controlling the power control module to supply power to the electrical devices in turn, according to the starting time sequence information.

The surgical robot may include a body and different external electrical devices. The power supply control method in this embodiment supplies power to different devices based on the power control module of the surgical robot. In a preferred embodiment, the power control module outputs DC power for supplying power by converting and controlling the input power.

In step S1, corresponding starting time sequence information is obtained for different electrical devices, and the time sequence information may be expressed by relative time (such as the startup sequence of several devices), programmed time (such as turning on a device when it reaches a certain moment) or their combination. On this basis, in step S2, the power control module is controlled to power on each electrical device in turn according to the starting time sequence, so as to realize power consumption peak shifting and avoid the corresponding over-current phenomenon. Of course, several electrical devices involved in the starting time sequence may be started at the same time, and there is no restriction that they must have a sequence, as long as they meet the requirements of over-current protection. Preferably, when the surgical robot is started, step S1 is automatically executed to start the detection and control of power supply. As shown in FIG. 2, it can be seen that the power supply system firstly meets the working requirements of the corresponding DC and AC conversion modules, and at the same time, the mechanical control system and related components may be powered on before the computer device.

As a preferred embodiment, the power control module includes at least one DC power control board and at least one AC power control board. The electrical devices include a DC electrical device and an AC electrical device. The DC power control board is configured to output DC power to a DC electrical device, and the AC power control board is configured to output AC power to an AC electrical device. Step S2 includes:

controlling the AC power control board and the DC power control board to supply power to the DC electrical device and the AC electrical device in turn, respectively; where, the starting time sequence information is set according to a working parameter of the DC electrical device and the AC electrical device, and the working parameter includes at least one of starting current and rated current.

When the power supply system is provided with the DC power output and the AC power output respectively, the types of the above-mentioned power outputs are distinguished and corresponding control is carried out in combination with the types of corresponding electrical device, so that the starting time sequence of the electrical device may be determined according to the working parameter such as the starting current and rated current, so as to ensure a stable output under the condition that the power supply system corresponds to a variety of different devices. As a preferred embodiment, the DC electrical device includes mechanical components corresponding to the surgical robot. The AC electrical device includes a computer device corresponding to the surgical robot.

In step S2, an order of supplying power to the DC electrical device and the AC electrical device in turn is to supply power to the computer device first, and then to the corresponding electric equipment and motorized device.

Specifically, a power transmission control is carried out for two types of electrical devices that are often involved in the application scenarios of surgical robots. First, power is supplied to the computer device, so that relevant control instructions can be issued, and then power is supplied to the corresponding electric equipment and motorized device, so that various application requirements in the robot surgery environment can be met.

The power supply control method for the surgical robot in this embodiment is based on the analysis of different electrical components of the surgical robot, and the corresponding electrical control logic is reasonably designed, so that the components of the surgical robot system can obtain independent DC power supply. By controlling the power-on time of the electrical components, the current surge can be effectively prevented when the electrical components are powered on, and the surgical robot can effectively work safely and stably, which has the significance of industry promotion.

### Embodiment 2

As shown in FIG. 3, this embodiment specifically provides a power supply control system for a surgical robot. The surgical robot includes a power control module for outputting power, and the control system includes:
an information reading module 1, configured to read starting time sequence information of corresponding electrical devices of the surgical robot; and
a power supply control module 2, configured to control the power control module to supply power to the electrical devices in turn according to the starting time sequence information.

The surgical robot may include a body and different external electrical devices. The power supply control method in this embodiment supplies power to different devices based on the power control module of the surgical robot. In a preferred embodiment, the power control module outputs DC power for supplying power by converting and controlling the input power.

The information reading module 1 obtains corresponding starting time sequence information for different electrical devices, and the time sequence information may be expressed by relative time (such as the startup sequence of several devices), programmed time (such as turning on a device when it reaches a certain moment) or their combination. On this basis, the power supply control module 2 controls the power control module to power on each electrical device in turn according to the starting time sequence, so as to realize power consumption peak shifting and avoid a corresponding over-current phenomenon. Of course, several electrical devices involved in the starting time sequence may be started at the same time, and there is no restriction that they must have a sequence, as long as they meet the requirements of over-current protection. Preferably, when the surgical robot is started, the information reading module 1 is automatically called to start the detection and control of power supply.

As a preferred embodiment, the power control module includes at least one DC power control board and at least one AC power control board. The electrical devices include a DC electrical device and an AC electrical device. The DC power control board is configured to output DC power to the DC electrical device, and the AC power control board is configured to output AC power to the AC electrical device. The power supply control module 2 is specifically configured to:
control the AC power control board and the DC power control board respectively, to supply power to the DC electrical device and the AC electrical device in turn; and the starting time sequence information is set according to a working parameter of the DC electrical device and the AC electrical device, and the working parameter includes at least one of starting current and rated current.

When the power supply system is provided with a DC power output and an AC power output respectively, this embodiment distinguishes types of the above power supply outputs and carries out corresponding control in combination with types of the corresponding electrical devices. Specifically, the starting time sequence of the electrical devices may be determined according to the working parameter such as the starting current and rated current, so as to ensure stable output when the power supply system corresponds to a variety of different devices.

As a preferred embodiment, the DC electrical device includes motor equipment corresponding to the surgical robot. The AC electrical device includes a computer device corresponding to the surgical robot.

An order of the power supply control module 2 supplying power to the DC electrical device and the AC electrical device in turn is as follows: firstly, supplying power to the computer device, and then supplying power to the corresponding electric equipment and motorized device.

In this embodiment, the power supply control is specifically carried out for two types of electrical devices that are often involved in the application scenario of the surgical robot. Firstly, the requirements of the relevant mechanical components of the robot are satisfied, which are usually more in line with the application requirements in the surgical environment. At the same time, the power input of computer device usually has more choices, so the adjustable range is wider.

The power supply control system for the surgical robot in this embodiment rationally designs the corresponding power control logic based on the analysis of different electrical components of the surgical robot, so that the components of the surgical robot system can obtain independent DC power supply. By controlling the power-on time of the electrical components, the current surge can be effectively prevented when the electrical components are powered on, so that the surgical robot can effectively work safely and stably, and the present disclosure has the significance of industry promotion.

### Embodiment 3

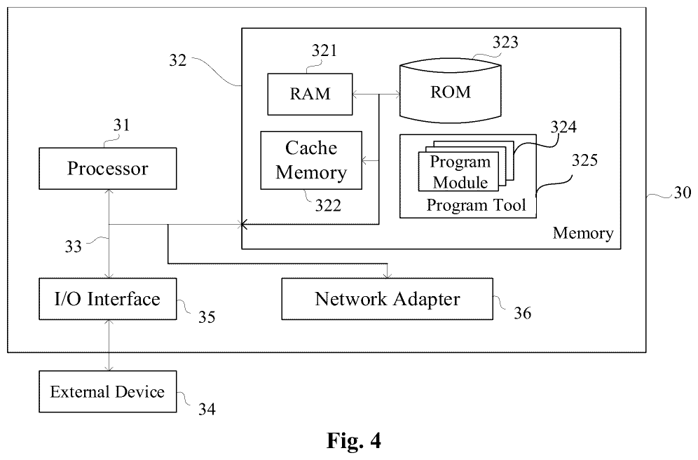

FIG. 4 is a schematic structural diagram of an electronic device provided by this embodiment. The electronic device includes a memory, a processor and a computer program stored in the memory and executable on the processor, and when the computer program is executed by the processor, the power supply control method for the surgical robot in the above embodiment is realized. The electronic device 30 shown in FIG. 4 is only an example, and should not bring any limitation to the function and application scope of the embodiment of the present disclosure.

As shown in FIG. 4, the electronic device 30 may be represented in the form of a general-purpose computing device, which may be a server device, for example. Components of the electronic device 30 may include, but are not limited to, the above-mentioned at least one processor 31, the above-mentioned at least one memory 32, and a bus 33 connecting different system components (including the memory 32 and the processor 31).

The bus 33 includes a data bus, an address bus and a control bus.

The memory 32 may include a volatile memory, such as a random-access memory (RAM) 321 and/or a cache memory 322, and may further include a read-only memory (ROM) 323.

The memory 32 may also include a program/utility 325 with a set of (at least one) program modules 324, such program modules 324 include, but are not limited to, an operating system, one or more application programs, other program modules and program data, and each or some combination of these examples may include the implementation of a network environment.

The processor 31 executes various functional applications and data processing by running computer programs stored in the memory 32, such as the power supply control method for the surgical robot as described above in the present disclosure.

The electronic device 30 may also communicate with one or more external devices 34 (e.g., keyboard, pointing device, etc.). This communication may be performed through an input/output (I/O) interface 35. Moreover, the device 30 generated by a model may also communicate with one or more networks (e.g., local area network (LAN), wide area network (WAN) and/or public network, such as the Internet) through a network adapter 36. As shown in FIG. 4, the network adapter 36 communicates with other modules of the device 30 generated by the model through the bus 33. It should be understood that although not shown in the figure, other hardware and/or software modules may be used in conjunction with the device 30 generated by the model, including but not limited to microcode, a device driver, a redundant processor, an external disk drive array, a redundant array of independent disks (RAID) system, a tape driver and a data backup storage system.

It should be noted that although several units/modules or sub-units/modules of the electronic device are mentioned in the above detailed description, this division is only exemplary and not mandatory. Actually, according to the embodiment of the present disclosure, the features and functions of two or more units/modules described above may be embodied in one unit/module. On the contrary, the features and functions of one unit/module described above may be further divided and embodied by multiple units/modules.

### Embodiment 4

This embodiment further provides a computer-readable storage medium, on which a computer program is stored, and when executed by a processor, the computer program realizes the steps in the power supply control method for the surgical robot of the above embodiment. The readable storage medium may be a portable disk, a hard disk, a random-access memory, a read-only memory, an erasable programmable read-only memory, an optical storage device, a magnetic storage device or any suitable combination of the above.

In a possible embodiment, the present disclosure may also be realized in the form of a program product, which includes a program code, and when the program product is run on a terminal device, the program code is used to make the terminal device execute the steps in the power supply control method for the surgical robot as described above. The program code for executing the present disclosure may be written in any combination of one or more programming languages, and the program may be completely executed on a user device, partially executed on the user device, executed as an independent software package, partially executed on the user device and partially executed on a remote device, or completely executed on the remote device.

Although specific embodiments of the present disclosure have been described above, those skilled in the art should understand that these are merely illustrative, and the scope of protection of the present disclosure is defined by the appended claims. Those skilled in the art may make many changes or modifications to these embodiments without departing from the principle and substance of the present disclosure, but these changes and modifications all fall within the protection scope of the present disclosure.

1. A power supply control method for a surgical robot, wherein the surgical robot comprises a power control module for outputting power, the control method comprising:
reading starting time sequence information of corresponding electrical devices of the surgical robot; and
controlling the power control module to supply power to the electrical devices in turn, according to the starting time sequence information.

2. The power supply control method for the surgical robot according to claim 1, wherein the power control module comprises at least one direct current (DC) power control board and at least one alternating current (AC) power control board; the electrical devices comprise a DC electrical device and an AC electrical device; the DC power control board is configured to output DC power to the DC electrical device, and the AC power control board is configured to output AC power to the AC electrical device; and the step of controlling the power control module to supply power to the electrical devices in turn comprises:
controlling the AC power control board and the DC power control board respectively, to supply power to the DC electrical device and the AC electrical device in turn; wherein the starting time sequence information is set according to a working parameter of the DC electrical device and the AC electrical device, and the working parameter comprises at least one of starting current and rated current.

3. The power supply control method for the surgical robot according to claim 2, wherein the DC electrical device comprises electric equipment corresponding to the surgical robot; and the AC electrical device comprises a computer device corresponding to the surgical robot; and
an order of supplying power to the DC electrical device and the AC electrical device in turn is as follows: firstly, supplying power to the computer device, and then supplying power to the corresponding electric equipment and a motorized device.

4. The power supply control method for the surgical robot according to claim 3,
wherein after the surgical robot is started, performing the step of reading the starting time sequence information of the corresponding electrical devices of the surgical robot.

5. A power supply control system for a surgical robot, wherein the surgical robot comprises a power control module for outputting power, and the control system comprises:
an information reading module, configured to read starting time sequence information of corresponding electrical devices of the surgical robot; and
a power supply control module, configured to control the power control module to supply power to the electrical devices in turn according to the starting time sequence information.

6. The power supply control system for the surgical robot according to claim 5, wherein the power control module comprises at least one DC power control board and at least one AC power control board; the electrical devices comprise a DC electrical device and an AC electrical device; the DC power control board is configured to output DC power to the DC electrical device, and the AC power control board is configured to output AC power to the AC electrical device; and
the power supply control module is configured to control the AC power control board and the DC power control board respectively, to supply power to the DC electrical device and the AC electrical device in turn; wherein the starting time sequence information is set according to a working parameter of the DC electrical device and the AC electrical device, and the working parameter comprises at least one of starting current and rated current.

7. The power supply control system for the surgical robot according to claim 6, wherein the DC electrical device comprises corresponding electric equipment corresponding to the surgical robot; and the AC electrical device comprises a computer device corresponding to the surgical robot; and
an order of the power supply control module supplying power to the DC electrical device and the AC electrical device in turn is as follows: firstly, supplying power to the computer device, and then supplying power to the corresponding electric equipment and a motorized device.

8. The power supply control system for the surgical robot according to claim 7, wherein the information reading module is called after the surgical robot is started.

9. An electronic device, comprising a memory, a processor and a computer program stored in the memory and executable on the processor, wherein in response to that the computer program is executed by the processor, the power supply control method for the surgical robot according to claim 1 is realized.

10. A computer-readable storage medium on which a computer program is stored, wherein in response to that the computer program is executed by a processor, the power supply control method for the surgical robot according to claim 1 is realized.
